(19) United States
(12) Patent Application Publication
(10) Pub. No.: US 2026/0337169 A1
(43) Pub. Date: Oct. 29, 2026

(54) POLYMORPH FORMS OF REBOXETINE

(71) Applicant: Axsome Therapeutics, Inc., New York, NY (US)

(72) Inventors: Herriot Tabuteau, New York, NY (US); Karen L. TenHuisen, Mountain Lakes, NJ (US); Patrick O’Hara, Madison, NJ (US); Ranjan Banerjee, Newton, MA (US); Jesus Juarez-Brambila, Jemison, AL (US)

(21) Appl. No.: 19/445,412

(22) Filed: Jan. 9, 2026

Related U.S. Application Data
(63) Continuation of application No. 19/096,360, filed on Mar. 31, 2025, now abandoned.
(60) Provisional application No. 63/738,431, filed on Dec. 23, 2024.

(51) Int. Cl. C07D 265/30; A61K 31/5375; A61K 47/02; A61K 47/32; A61K 47/38

(52) U.S. Cl. CPC C07D 265/30; A61K 31/5375; A61K 47/02; A61K 47/32; A61K 47/38

(57) ABSTRACT

Polymorph forms of reboxetine, and their therapeutic uses, are described herein.

### CROSS-REFERENCE TO RELATED APPLICATIONS

This application is a continuation of U.S. patent application Ser. No. 19/096,360, filed Mar. 31, 2025, which claims the priority to U.S. Provisional Patent Application No. 63/738,431, filed Dec. 23, 2024, both of which are incorporated by reference herein in their entirety.

### BACKGROUND

Narcolepsy is a serious and debilitating neurological condition that causes dysregulation of the sleep-wake cycle and is characterized clinically by excessive daytime sleepiness (EDS), cataplexy, hypnagogic hallucinations, sleep paralysis, and disrupted nocturnal sleep. Narcolepsy is estimated to afflict an estimated 185,000 individuals in the U.S. Cataplexy is seen in an estimated 70% of narcolepsy patients and is a sudden reduction or loss of muscle tone while a patient is awake, typically triggered by strong emotions such as laughter, fear, anger, stress, or excitement. Type 1 narcolepsy includes cataplexy, while Type 2 narcolepsy does not include cataplexy. Narcolepsy interferes with cognitive, psychological, and social functioning, increases the risk of work- and driving-related accidents, and is associated with a 1.5 fold higher mortality rate. Depression is reported in up to 57% of patients. Unfortunately, currently approved treatments are few for this under-diagnosed orphan condition and are limited by variability in efficacy from patient to patient, tolerability issues and the need for Drug Enforcement Administration (DEA) scheduling.

### SUMMARY

This disclosure relates generally to the treatment of narcolepsy, such as narcolepsy with cataplexy, or Type 1 narcolepsy. For example, a pharmaceutical composition for treating narcolepsy with cataplexy, for example, by reducing the number or frequency of cataplexy attacks, may comprise a therapeutically effective amount of reboxetine free base, or a pharmaceutically acceptable salt thereof, such as reboxetine mesylate, and a pharmaceutically acceptable carrier is disclosed.

One aspect of the present disclosure relates to reboxetine, such as reboxetine composition, for example, a reboxetine composition that is in a tablet dosage form.

Some embodiments include a pharmaceutical composition comprising a uniform blend of reboxetine and a pharmaceutically acceptable excipient.

Some embodiments include a polymorph of reboxetine having an X-Ray power diffractogram (XRPD) comprising a peak located at a position (° 2θ) of 7.63, 9.88, 11.45, 14.43, 14.73, 15.89, 17.05, 17.43, 18.04, 18.80, 19.36, 25.80, or a combination thereof.

Some embodiments include comprise compound represented by a formula:

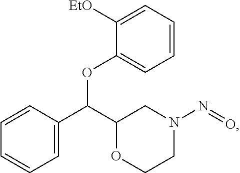 [SMILES: CCOc1ccccc1OC(c1ccccc1)C1CN(N=O)CCO1]

having a name 2-((2-ethoxyphenoxy)(phenyl)methyl)-4-nitrosomorpholine.

Some embodiments include a method of determining the quality of a pharmaceutical composition comprising reboxetine, comprising comparing a chromatographic result obtained from the pharmaceutical composition to a chromatographic result obtained from 2-((2-ethoxyphenoxy)(phenyl)methyl)-4-nitrosomorpholine.

Some embodiments include a dosage form reboxetine and less than 13 ppm of a compound represented by a formula:

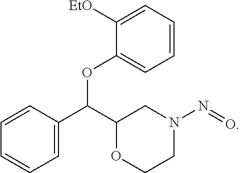 [SMILES: CCOc1ccccc1OC(c1ccccc1)C1CN(N=O)CCO1]

In some embodiments, the reboxetine comprises a polymorph of reboxetine that has an X-Ray power diffractogram (XRPD) comprising a peak located at a position (° 2θ) of 7.63, 9.88, 11.45, 14.43, 14.73, 15.89, 17.05, 17.43, 18.04, 18.80, 19.36, 25.80, or a combination thereof.

### BRIEF DESCRIPTION OF THE DRAWINGS

FIG. 1 depicts an X-Ray powder diffractogram (XRPD) of Polymorph 1.

FIG. 2 depicts an XRPD of Polymorph 2.

FIG. 3 depicts a Differential Scanning calorimetry (DSC) thermogram of Polymorph 1 at a 1 ° C/min heating rate.

FIG. 4 depicts a Differential Scanning calorimetry (DSC) thermogram of Polymorph 2 at a 1 ° C/min heating rate.

### DETAILED DESCRIPTION

The compositions or dosage forms described herein may be useful for treating a neurological condition, such as narcolepsy with cataplexy, and for other purposes.

Detailed descriptions of one or more embodiments are provided herein. It is to be understood, however, that the present disclosure is embodied in various forms. Therefore, specific details disclosed herein are not to be interpreted as limiting, but rather as a basis for the claims and as a representative basis for teaching one skilled in the art to employ the present disclosure in any appropriate manner.

### Reboxetine Tablet Dosage Forms

The dosage form, such as a tablet, may contain reboxetine free base, such as about 4-6 mg, about 4.5-5.5 mg, or about 5 mg of reboxetine free base, or a molar equivalent amount of a salt form of reboxetine, such as about 5.5-7.5 mg, about 6-7 mg, or about 6.5 mg of reboxetine mesylate. 6.5 mg reboxetine mesylate is equivalent to 5 mg reboxetine free base.

The dosage form, such as a tablet, may contain multiple layers, such as about 1 to 4 layers, about 1 to 3 layers, or about 2 layers. In some embodiments, the tablet may contain an intra granular component and an extra granular component. For example, the intra granular component may comprise an active ingredient, API, a diluent, a binder, and/or a disintegrant. For example, the extra granular component may comprise a glidant, a disintegrant, and/or a lubricant. In some embodiments, the intra granular component may be about 90-100%, about 93-97%, about 95-96%, or about 96.9% of the weight of the dosage form. In some embodiments, the extra granular component may be about 1-10%, about 2.5-5%, about 3-4%, or about 3.1% of the weight of the dosage form.

The dosage form, such as a tablet, may contain a diluent (such as microcrystalline cellulose and/or calcium phosphate), such as about 50-99%, about 75-95%, about 80-90%, or about 85-86% diluent by weight.

The dosage form, such as a tablet, may contain microcrystalline cellulose, as a binder and/or a diluent, such as about 50-80%, about 60-70%, about 63-66%, or about 64.6% microcrystalline cellulose by weight. In some embodiments, the microcrystalline cellulose is microcrystalline cellulose PH102.

The dosage form, such as a tablet, may contain calcium phosphate, such as calcium phosphate dibasic dihydrate. In some embodiments, the tablet contains about 10-30%, about 15-25%, about 20-22%, or about 21.5% of calcium phosphate dibasic dihydrate by weight.

The dosage form, such as a tablet, may contain a binder (such as microcrystalline cellulose and/or hydroxypropyl methylcellulose), such as about 2.5-10%, about 4-7.5%, about 5%, about 60-80%, or about 65-75% binder by weight.

The dosage form, such as a tablet, may contain hydroxypropyl methylcellulose (HPMC), also referred to as Hypromellose, such as about 2.5-10%, about 4-7.5%, or about 5% hydroxypropyl methylcellulose by weight. In some embodiments, the hydroxypropyl methylcellulose is Hypromellose 2910-5.

The dosage form, such as a tablet, may contain polyvinylpolypyrrolidone, also referred to as Crospovidone, such as about 1-10%, about 2-5%, or about 2.5% polyvinylpolypyrrolidone by weight. In some embodiments, the polyvinylpolypyrrolidone is Crospovidone Ultra 10.

The dosage form, such as a tablet, may contain silicone dioxide colloidal, such as about 0.01-1%, about 0.05-0.5%, or about 0.1% by weight.

The dosage form, such as a tablet, may contain magnesium stearate such as about 0.1-5%, about 0.5-2.5%, or about 0.5% by weight. In some embodiments, the magnesium stearate is Magnesium Stearate 5712.

The dosage form, such as a tablet, may be coated, e.g., with a film coating. For example, a film coating may comprise a polymer, a plasticizer, and/or a pigment. The film coating may be, e.g., about 1-10%, about 4-6%, or about 3.5% of the weight of the dosage form or tablet. In some embodiments, the film coating may be Opadry II Pink 85F140228-CN.

In some embodiments, the dosage form contains reboxetine free base, such as about 4-6 mg, about 4.5-5.5 mg, or about 5 mg of reboxetine free base, or a molar equivalent amount of a salt form of reboxetine, such as about 5.5-7.5 mg, about 6-7 mg, or about 6.5 mg of reboxetine mesylate; about 50-80%, about 60-70%, about 63-66%, or about 64.6% microcrystalline cellulose by weight; about 10-30%, about 15-25%, about 20-22%, or about 21.5% of calcium phosphate dibasic dihydrate by weight; about 2.5-10%, about 4-7.5%, or about 5% hydroxypropyl methylcellulose by weight; about 1-10%, about 2-5%, or about 2.5% polyvinylpolypyrrolidone by weight; about 0.01-1%, about 0.03-0.5%, or about 0.1% silicone dioxide colloidal by weight; and about 0.1-5%, about 0.3-2.5%, or about 0.5% magnesium stearate by weight.

In some embodiments, the dosage form contains reboxetine free base, such as about 4-6 mg, about 4.5-5.5 mg, or about 5 mg of reboxetine free base, or a molar equivalent amount of a salt form of reboxetine, such as about 5.5-7.5 mg, about 6-7 mg, or about 6.5 mg of reboxetine mesylate; about 50-80%, about 60-70%, about 63-66%, or about 64.6% microcrystalline cellulose by weight; about 10-30%, about 15-25%, about 20-22%, or about 21.5% of calcium phosphate dibasic dihydrate by weight; about 2.5-10%, about 4-7.5%, or about 5% hydroxypropyl methylcellulose by weight; about 1-10%, about 2-5%, or about 2.5% polyvinylpolypyrrolidone by weight; about 0.01-1%, about 0.03-0.5%, or about 0.1% silicone dioxide colloidal by weight; and the film coating may be, e.g., about 1-10%, about 4-6%, or about 3.5% of the weight of the dosage form or tablet.

In some embodiments, the dosage form contains reboxetine free base, such as about 4-6 mg, about 4.5-5.5 mg, or about 5 mg of reboxetine free base, or a molar equivalent amount of a salt form of reboxetine, such as about 5.5-7.5 mg, about 6-7 mg, or about 6.5 mg of reboxetine mesylate; about 10-30%, about 15-25%, about 20-22%, or about 21.5% of calcium phosphate dibasic dihydrate by weight; about 2.5-10%, about 4-7.5%, or about 5% hydroxypropyl methylcellulose by weight; about 1-10%, about 2-5%, or about 2.5% polyvinylpolypyrrolidone by weight; about 0.01-1%, about 0.03-0.5%, or about 0.1% silicone dioxide colloidal by weight; about 0.1-5%, about 0.3-2.5%, or about 0.5% magnesium stearate by weight; and the film coating may be, e.g., about 1-10%, about 4-6%, or about 3.5% of the weight of the dosage form or tablet.

In some embodiments, the dosage form contains reboxetine free base, such as 4-6 mg, about 4.5-5.5 mg, or about 5 mg of reboxetine free base, or a molar equivalent amount of a salt form of reboxetine, such as about 5.5-7.5 mg, about 6-7 mg, or about 6.5 mg of reboxetine mesylate; about 50-80%, about 60-70%, about 63-66%, or about 64.6% microcrystalline cellulose by weight; about 10-30%, about 15-25%, about 20-22%, or about 21.5% of calcium phosphate dibasic dihydrate by weight; about 2.5-10%, about 4-7.5%, or about 5% hydroxypropyl methylcellulose by weight; about 0.01-1%, about 0.03-0.5%, or about 0.1% silicone dioxide colloidal by weight; about 0.1-5%, about 0.3-2.5%, or about 0.5% magnesium stearate by weight; and the film coating may be, e.g., about 1-10%, about 4-6%, or about 3.5% of the weight of the dosage form or tablet.

In some embodiments, the dosage form contains reboxetine free base, such as about 4-6 mg, about 4.5-5.5 mg, or about 5 mg of reboxetine free base, or a molar equivalent amount of a salt form of reboxetine, such as about 5.5-7.5 mg, about 6-7 mg, or about 6.5 mg of reboxetine mesylate; about 10-30%, about 15-25%, about 20-22%, or about 21.5% of calcium phosphate dibasic dihydrate by weight; about 1-10%, about 2-5%, or about 2.5% polyvinylpolypyrrolidone by weight; about 50-80%, about 60-70%, about 63-66%, or about 64.6% microcrystalline cellulose by weight; about 0.01-1%, about 0.03-0.5%, or about 0.1% silicone dioxide colloidal by weight; about 0.1-5%, about 0.3-2.5%, or about 0.5% magnesium stearate by weight; and the film coating may be, e.g., about 1-10%, about 4-6%, or about 3.5% of the weight of the dosage form or tablet.

In some embodiments, the dosage form contains reboxetine free base, such as about 4-6 mg, about 4.5-5.5 mg, or about 5 mg of reboxetine free base, or a molar equivalent amount of a salt form of reboxetine, such as about 5.5-7.5 mg, about 6-7 mg, or about 6.5 mg of reboxetine mesylate; about 2.5-10%, about 4-7.5%, or about 5% hydroxypropyl methylcellulose by weight; about 1-10%, about 2-5%, or about 2.5% polyvinylpolypyrrolidone by weight; about 50-80%, about 60-70%, about 63-66%, or about 64.6% microcrystalline cellulose by weight; about 0.01-1%, about 0.03-0.5%, or about 0.1% silicone dioxide colloidal by weight; about 0.1-5%, about 0.3-2.5%, or about 0.5% magnesium stearate by weight; and the film coating may be, e.g., about 1-10%, about 4-6%, or about 3.5% of the weight of the dosage form or tablet.

In some embodiments, the dosage form contains reboxetine free base, such as about 4-6 mg, about 4.5-5.5 mg, or about 5 mg of reboxetine free base, or a molar equivalent amount of a salt form of reboxetine, such as about 5.5-7.5 mg, about 6-7 mg, or about 6.5 mg of reboxetine mesylate; about 50-80%, about 60-70%, about 63-66%, or about 64.6% microcrystalline cellulose by weight; about 10-30%, about 15-25%, about 20-22%, or about 21.5% of calcium phosphate dibasic dihydrate by weight; about 2.5-10%, about 4-7.5%, or about 5% hydroxypropyl methylcellulose by weight; about 1-10%, about 2-5%, or about 2.5% polyvinylpolypyrrolidone by weight; and about 0.1-5%, about 0.3-2.5%, or about 0.5% magnesium stearate by weight.

In some embodiments, the dosage form contains reboxetine free base, such as about 4-6 mg, about 4.5-5.5 mg, or about 5 mg of reboxetine free base, or a molar equivalent amount of a salt form of reboxetine, such as about 5.5-7.5 mg, about 6-7 mg, or about 6.5 mg of reboxetine mesylate; about 50-80%, about 60-70%, about 63-66%, or about 64.6% microcrystalline cellulose by weight; about 10-30%, about 15-25%, about 20-22%, or about 21.5% of calcium phosphate dibasic dihydrate by weight; about 2.5-10%, about 4-7.5%, or about 5% hydroxypropyl methylcellulose by weight; about 0.01-1%, about 0.03-0.5%, or about 0.1% silicone dioxide colloidal by weight; and about 0.1-5%, about 0.3-2.5%, or about 0.5% magnesium stearate by weight.

In some embodiments, the dosage form contains reboxetine free base, such as about 4-6 mg, about 4.5-5.5 mg, or about 5 mg of reboxetine free base, or a molar equivalent amount of a salt form of reboxetine, such as about 5.5-7.5 mg, about 6-7 mg, or about 6.5 mg of reboxetine mesylate; about 50-80%, about 60-70%, about 63-66%, or about 64.6% microcrystalline cellulose by weight; about 10-30%, about 15-25%, about 20-22%, or about 21.5% of calcium phosphate dibasic dihydrate by weight; about 1-10%, about 2-5%, or about 2.5% polyvinylpolypyrrolidone by weight; about 0.01-1%, about 0.03-0.5%, or about 0.1% silicone dioxide colloidal by weight; and about 0.1-5%, about 0.3-2.5%, or about 0.5% magnesium stearate by weight.

In some embodiments, the dosage form contains reboxetine free base, such as about 4-6 mg, about 4.5-5.5 mg, or about 5 mg of reboxetine free base, or a molar equivalent amount of a salt form of reboxetine, such as about 5.5-7.5 mg, about 6-7 mg, or about 6.5 mg of reboxetine mesylate; about 50-80%, about 60-70%, about 63-66%, or about 64.6% microcrystalline cellulose by weight; about 2.5-10%, about 4-7.5%, or about 5% hydroxypropyl methylcellulose by weight; about 1-10%, about 2-5%, or about 2.5% polyvinylpolypyrrolidone by weight; about 0.01-1%, about 0.03-0.5%, or about 0.1% silicone dioxide colloidal by weight; and about 0.1-5%, about 0.3-2.5%, or about 0.5% magnesium stearate by weight.

In some embodiments, the dosage form contains reboxetine free base, such as 4-6 mg, about 4.5-5.5 mg, or about 5 mg of reboxetine free base, or a molar equivalent amount of a salt form of reboxetine, such as about 5.5-7.5 mg, about 6-7 mg, or about 6.5 mg of reboxetine mesylate; about 10-30%, about 15-25%, about 20-22%, or about 21.5% of calcium phosphate dibasic dihydrate by weight; about 2.5-10%, about 4-7.5%, or about 5% hydroxypropyl methylcellulose by weight; about 1-10%, about 2-5%, or about 2.5% polyvinylpolypyrrolidone by weight; about 0.01-1%, about 0.03-0.5%, or about 0.1% silicone dioxide colloidal by weight; and about 0.1-5%, about 0.3-2.5%, or about 0.5% magnesium stearate by weight.

In some embodiments, the dosage form contains reboxetine free base, such as about 4-6 mg, about 4.5-5.5 mg, or about 5 mg of reboxetine free base, or a molar equivalent amount of a salt form of reboxetine, such as about 5.5-7.5 mg, about 6-7 mg, or about 6.5 mg of reboxetine mesylate; about 50-80%, about 60-70%, about 63-66%, or about 64.6% microcrystalline cellulose by weight; about 10-30%, about 15-25%, about 20-22%, or about 21.5% of calcium phosphate dibasic dihydrate by weight; about 2.5-10%, about 4-7.5%, or about 5% hydroxypropyl methylcellulose by weight; about 1-10%, about 2-5%, or about 2.5% polyvinylpolypyrrolidone by weight; and the film coating may be, e.g., about 1-10%, about 4-6%, or about 3.5% of the weight of the dosage form or tablet.

In some embodiments, the dosage form contains reboxetine free base, such as about 4-6 mg, about 4.5-5.5 mg, or about 5 mg of reboxetine free base, or a molar equivalent amount of a salt form of reboxetine, such as about 5.5-7.5 mg, about 6-7 mg, or about 6.5 mg of reboxetine mesylate; about 50-80%, about 60-70%, about 63-66%, or about 64.6% microcrystalline cellulose by weight; about 10-30%, about 15-25%, about 20-22%, or about 21.5% of calcium phosphate dibasic dihydrate by weight; about 2.5-10%, about 4-7.5%, or about 5% hydroxypropyl methylcellulose by weight; about 0.01-1%, about 0.03-0.5%, or about 0.1% silicone dioxide colloidal by weight; and the film coating may be, e.g., about 1-10%, about 4-6%, or about 3.5% of the weight of the dosage form or tablet.

In some embodiments, the dosage form contains reboxetine free base, such as about 4-6 mg, about 4.5-5.5 mg, or about 5 mg of reboxetine free base, or a molar equivalent amount of a salt form of reboxetine, such as about 5.5-7.5 mg, about 6-7 mg, or about 6.5 mg of reboxetine mesylate; about 50-80%, about 60-70%, about 63-66%, or about 64.6% microcrystalline cellulose by weight; about 10-30%, about 15-25%, about 20-22%, or about 21.5% of calcium phosphate dibasic dihydrate by weight; about 1-10%, about 2-5%, or about 2.5% polyvinylpolypyrrolidone by weight; about 0.01-1%, about 0.03-0.5%, or about 0.1% silicone dioxide colloidal by weight; and the film coating may be, e.g., about 1-10%, about 4-6%, or about 3.5% of the weight of the dosage form or tablet.

In some embodiments, the dosage form contains reboxetine free base, such as about 4-6 mg, about 4.5-5.5 mg, or about 5 mg of reboxetine free base, or a molar equivalent amount of a salt form of reboxetine, such as about 5.5-7.5 mg, about 6-7 mg, or about 6.5 mg of reboxetine mesylate; about 50-80%, about 60-70%, about 63-66%, or about 64.6% microcrystalline cellulose by weight; about 2.5-10%, about 4-7.5%, or about 5% hydroxypropyl methylcellulose by weight; about 1-10%, about 2-5%, or about 2.5% polyvinylpolypyrrolidone by weight; about 0.01-1%, about 0.03-0.5%, or about 0.1% silicone dioxide colloidal by weight; and the film coating may be, e.g., about 1-10%, about 4-6%, or about 3.5% of the weight of the dosage form or tablet.

In some embodiments, the dosage form contains reboxetine free base, such as about 4-6 mg, about 4.5-5.5 mg, or about 5 mg of reboxetine free base, or a molar equivalent amount of a salt form of reboxetine, such as about 5.5-7.5 mg, about 6-7 mg, or about 6.5 mg of reboxetine mesylate; about 10-30%, about 15-25%, about 20-22%, or about 21.5% of calcium phosphate dibasic dihydrate by weight; about 2.5-10%, about 4-7.5%, or about 5% hydroxypropyl methylcellulose by weight; about 1-10%, about 2-5%, or about 2.5% polyvinylpolypyrrolidone by weight; about 0.01-1%, about 0.03-0.5%, or about 0.1% silicone dioxide colloidal by weight; and the film coating may be, e.g., about 1-10%, about 4-6%, or about 3.5% of the weight of the dosage form or tablet.

In some embodiments, the dosage form contains reboxetine free base, such as about 4-6 mg, about 4.5-5.5 mg, or about 5 mg of reboxetine free base, or a molar equivalent amount of a salt form of reboxetine, such as about 5.5-7.5 mg, about 6-7 mg, or about 6.5 mg of reboxetine mesylate; about 50-80%, about 60-70%, about 63-66%, or about 64.6% microcrystalline cellulose by weight; about 10-30%, about 15-25%, about 20-22%, or about 21.5% of calcium phosphate dibasic dihydrate by weight; about 2.5-10%, about 4-7.5%, or about 5% hydroxypropyl methylcellulose by weight; about 0.1-5%, about 0.3-2.5%, or about 0.5% magnesium stearate by weight; and the film coating may be, e.g., about 1-10%, about 4-6%, or about 3.5% of the weight of the dosage form or tablet.

In some embodiments, the dosage form contains reboxetine free base, such as about 4-6 mg, about 4.5-5.5 mg, or about 5 mg of reboxetine free base, or a molar equivalent amount of a salt form of reboxetine, such as about 5.5-7.5 mg, about 6-7 mg, or about 6.5 mg of reboxetine mesylate; about 50-80%, about 60-70%, about 63-66%, or about 64.6% microcrystalline cellulose by weight; about 10-30%, about 15-25%, about 20-22%, or about 21.5% of calcium phosphate dibasic dihydrate by weight; about 1-10%, about 2-5%, or about 2.5% polyvinylpolypyrrolidone by weight; about 0.1-5%, about 0.3-2.5%, or about 0.5% magnesium stearate by weight; and the film coating may be, e.g., about 1-10%, about 4-6%, or about 3.5% of the weight of the dosage form or tablet.

In some embodiments, the dosage form contains reboxetine free base, such as about 4-6 mg, about 4.5-5.5 mg, or about 5 mg of reboxetine free base, or a molar equivalent amount of a salt form of reboxetine, such as about 5.5-7.5 mg, about 6-7 mg, or about 6.5 mg of reboxetine mesylate; about 50-80%, about 60-70%, about 63-66%, or about 64.6% microcrystalline cellulose by weight; about 2.5-10%, about 4-7.5%, or about 5% hydroxypropyl methylcellulose by weight; about 1-10%, about 2-5%, or about 2.5% polyvinylpolypyrrolidone by weight; about 0.1-5%, about 0.3-2.5%, or about 0.5% magnesium stearate by weight; and the film coating may be, e.g., about 1-10%, about 4-6%, or about 3.5% of the weight of the dosage form or tablet.

In some embodiments, the dosage form contains reboxetine free base, such as about 4-6 mg, about 4.5-5.5 mg, or about 5 mg of reboxetine free base, or a molar equivalent amount of a salt form of reboxetine, such as about 5.5-7.5 mg, about 6-7 mg, or about 6.5 mg of reboxetine mesylate; about 10-30%, about 15-25%, about 20-22%, or about 21.5% of calcium phosphate dibasic dihydrate by weight; about 2.5-10%, about 4-7.5%, or about 5% hydroxypropyl methylcellulose by weight; about 1-10%, about 2-5%, or about 2.5% polyvinylpolypyrrolidone by weight; 0.1-5%, about 0.3-2.5%, or about 0.5% magnesium stearate by weight; and the film coating may be, e.g., about 1-10%, about 4-6%, or about 3.5% of the weight of the dosage form or tablet.

In some embodiments, the dosage form contains reboxetine free base, such as about 4-6 mg, about 4.5-5.5 mg, or about 5 mg of reboxetine free base, or a molar equivalent amount of a salt form of reboxetine, such as about 5.5-7.5 mg, about 6-7 mg, or about 6.5 mg of reboxetine mesylate; about 50-80%, about 60-70%, about 63-66%, or about 64.6% microcrystalline cellulose by weight; about 10-30%, about 15-25%, about 20-22%, or about 21.5% of calcium phosphate dibasic dihydrate by weight; about 0.01-1%, about 0.03-0.5%, or about 0.1% silicone dioxide colloidal by weight; about 0.1-5%, about 0.3-2.5%, or about 0.5% magnesium stearate by weight; and the film coating may be, e.g., about 1-10%, about 4-6%, or about 3.5% of the weight of the dosage form or tablet.

In some embodiments, the dosage form contains reboxetine free base, such as about 4-6 mg, about 4.5-5.5 mg, or about 5 mg of reboxetine free base, or a molar equivalent amount of a salt form of reboxetine, such as about 5.5-7.5 mg, about 6-7 mg, or about 6.5 mg of reboxetine mesylate; about 50-80%, about 60-70%, about 63-66%, or about 64.6% microcrystalline cellulose by weight; about 2.5-10%, about 4-7.5%, or about 5% hydroxypropyl methylcellulose by weight; about 0.01-1%, about 0.03-0.5%, or about 0.1% silicone dioxide colloidal by weight; about 0.1-5%, about 0.3-2.5%, or about 0.5% magnesium stearate by weight; and the film coating may be, e.g., about 1-10%, about 4-6%, or about 3.5% of the weight of the dosage form or tablet.

In some embodiments, the dosage form contains reboxetine free base, such as about 4-6 mg, about 4.5-5.5 mg, or about 5 mg of reboxetine free base, or a molar equivalent amount of a salt form of reboxetine, such as about 5.5-7.5 mg, about 6-7 mg, or about 6.5 mg of reboxetine mesylate; about 10-30%, about 15-25%, about 20-22%, or about 21.5% of calcium phosphate dibasic dihydrate by weight; about 2.5-10%, about 4-7.5%, or about 5% hydroxypropyl methylcellulose by weight; about 0.01-1%, about 0.03-0.5%, or about 0.1% silicone dioxide colloidal by weight; about 0.1-5%, about 0.3-2.5%, or about 0.5% magnesium stearate by weight; and the film coating may be, e.g., about 1-10%, about 4-6%, or about 3.5% of the weight of the dosage form or tablet.

In some embodiments, the dosage form contains reboxetine free base, such as about 4-6 mg, about 4.5-5.5 mg, or about 5 mg of reboxetine free base, or a molar equivalent amount of a salt form of reboxetine, such as about 5.5-7.5 mg, about 6-7 mg, or about 6.5 mg of reboxetine mesylate; about 50-80%, about 60-70%, about 63-66%, or about 64.6% microcrystalline cellulose by weight; about 1-10%, about 2-5%, or about 2.5% polyvinylpolypyrrolidone by weight; about 0.01-1%, about 0.03-0.5%, or about 0.1% silicone dioxide colloidal by weight; about 0.1-5%, about 0.3-2.5%, or about 0.5% magnesium stearate by weight; and the film coating may be, e.g., about 1-10%, about 4-6%, or about 3.5% of the weight of the dosage form or tablet.

In some embodiments, the dosage form contains reboxetine free base, such as about 4-6 mg, about 4.5-5.5 mg, or about 5 mg of reboxetine free base, or a molar equivalent amount of a salt form of reboxetine, such as about 5.5-7.5 mg, about 6-7 mg, or about 6.5 mg of reboxetine mesylate; about 10-30%, about 15-25%, about 20-22%, or about 21.5% of calcium phosphate dibasic dihydrate by weight; about 1-10%, about 2-5%, or about 2.5% polyvinylpolypyrrolidone by weight; about 0.01-1%, about 0.03-0.5%, or about 0.1% silicone dioxide colloidal by weight;

about 0.1-5%, about 0.3-2.5%, or about 0.5% magnesium stearate by weight; and the film coating may be, e.g., about 1-10%, about 4-6%, or about 3.5% of the weight of the dosage form or tablet.

In some embodiments, the dosage form contains reboxetine free base, such as about 4-6 mg, about 4.5-5.5 mg, or about 5 mg of reboxetine free base, or a molar equivalent amount of a salt form of reboxetine, such as about 5.5-7.5 mg, about 6-7 mg, or about 6.5 mg of reboxetine mesylate; about 50-80%, about 60-70%, about 63-66%, or about 64.6% microcrystalline cellulose by weight; about 10-30%, about 15-25%, about 20-22%, or about 21.5% of calcium phosphate dibasic dihydrate by weight; about 2.5-10%, about 4-7.5%, or about 5% hydroxypropyl methylcellulose by weight; and the film coating may be, e.g., about 1-10%, about 4-6%, or about 3.5% of the weight of the dosage form or tablet.

In some embodiments, the dosage form contains reboxetine free base, such as about 4-6 mg, about 4.5-5.5 mg, or about 5 mg of reboxetine free base, or a molar equivalent amount of a salt form of reboxetine, such as about 5.5-7.5 mg, about 6-7 mg, or about 6.5 mg of reboxetine mesylate; about 50-80%, about 60-70%, about 63-66%, or about 64.6% microcrystalline cellulose by weight; about 10-30%, about 15-25%, about 20-22%, or about 21.5% of calcium phosphate dibasic dihydrate by weight; about 1-10%, about 2-5%, or about 2.5% polyvinylpolypyrrolidone by weight; and the film coating may be, e.g., about 1-10%, about 4-6%, or about 3.5% of the weight of the dosage form or tablet.

In some embodiments, the dosage form contains reboxetine free base, such as about 4-6 mg, about 4.5-5.5 mg, or about 5 mg of reboxetine free base, or a molar equivalent amount of a salt form of reboxetine, such as about 5.5-7.5 mg, about 6-7 mg, or about 6.5 mg of reboxetine mesylate; about 50-80%, about 60-70%, about 63-66%, or about 64.6% microcrystalline cellulose by weight; about 2.5-10%, about 4-7.5%, or about 5% hydroxypropyl methylcellulose by weight; about 1-10%, about 2-5%, or about 2.5% polyvinylpolypyrrolidone by weight; and the film coating may be, e.g., about 1-10%, about 4-6%, or about 3.5% of the weight of the dosage form or tablet.

In some embodiments, the dosage form contains reboxetine free base, such as about 4-6 mg, about 4.5-5.5 mg, or about 5 mg of reboxetine free base, or a molar equivalent amount of a salt form of reboxetine, such as about 5.5-7.5 mg, about 6-7 mg, or about 6.5 mg of reboxetine mesylate; about 10-30%, about 15-25%, about 20-22%, or about 21.5% of calcium phosphate dibasic dihydrate by weight; about 2.5-10%, about 4-7.5%, or about 5% hydroxypropyl methylcellulose by weight; about 1-10%, about 2-5%, or about 2.5% polyvinylpolypyrrolidone by weight; and the film coating may be, e.g., about 1-10%, about 4-6%, or about 3.5% of the weight of the dosage form or tablet.

In some embodiments, the dosage form contains reboxetine free base, such as about 4-6 mg, about 4.5-5.5 mg, or about 5 mg of reboxetine free base, or a molar equivalent amount of a salt form of reboxetine, such as about 5.5-7.5 mg, about 6-7 mg, or about 6.5 mg of reboxetine mesylate; about 50-80%, about 60-70%, about 63-66%, or about 64.6% microcrystalline cellulose by weight; about 10-30%, about 15-25%, about 20-22%, or about 21.5% of calcium phosphate dibasic dihydrate by weight; about 2.5-10%, about 4-7.5%, or about 5% hydroxypropyl methylcellulose by weight; and about 0.01-1%, about 0.03-0.5%, or about 0.1% silicone dioxide colloidal by weight.

In some embodiments, the dosage form contains reboxetine free base, such as about 4-6 mg, about 4.5-5.5 mg, or about 5 mg of reboxetine free base, or a molar equivalent amount of a salt form of reboxetine, such as about 5.5-7.5 mg, about 6-7 mg, or about 6.5 mg of reboxetine mesylate; about 50-80%, about 60-70%, about 63-66%, or about 64.6% microcrystalline cellulose by weight; about 10-30%, about 15-25%, about 20-22%, or about 21.5% of calcium phosphate dibasic dihydrate by weight; about 1-10%, about 2-5%, or about 2.5% polyvinylpolypyrrolidone by weight; and about 0.01-1%, about 0.03-0.5%, or about 0.1% silicone dioxide colloidal by weight.

In some embodiments, the dosage form contains reboxetine free base, such as about 4-6 mg, about 4.5-5.5 mg, or about 5 mg of reboxetine free base, or a molar equivalent amount of a salt form of reboxetine, such as about 5.5-7.5 mg, about 6-7 mg, or about 6.5 mg of reboxetine mesylate; about 50-80%, about 60-70%, about 63-66%, or about 64.6% microcrystalline cellulose by weight; about 2.5-10%, about 4-7.5%, or about 5% hydroxypropyl methylcellulose by weight; about 1-10%, about 2-5%, or about 2.5% polyvinylpolypyrrolidone by weight; and about 0.01-1%, about 0.03-0.5%, or about 0.1% silicone dioxide colloidal by weight.

In some embodiments, the dosage form contains reboxetine free base, such as about 4-6 mg, about 4.5-5.5 mg, or about 5 mg of reboxetine free base, or a molar equivalent amount of a salt form of reboxetine, such as about 5.5-7.5 mg, about 6-7 mg, or about 6.5 mg of reboxetine mesylate; about 10-30%, about 15-25%, about 20-22%, or about 21.5% of calcium phosphate dibasic dihydrate by weight; about 2.5-10%, about 4-7.5%, or about 5% hydroxypropyl methylcellulose by weight; about 1-10%, about 2-5%, or about 2.5% polyvinylpolypyrrolidone by weight; and about 0.01-1%, about 0.03-0.5%, or about 0.1% silicone dioxide colloidal by weight.

In some embodiments, the dosage form contains reboxetine free base, such as about 4-6 mg, about 4.5-5.5 mg, or about 5 mg of reboxetine free base, or a molar equivalent amount of a salt form of reboxetine, such as about 5.5-7.5 mg, about 6-7 mg, or about 6.5 mg of reboxetine mesylate; about 50-80%, about 60-70%, about 63-66%, or about 64.6% microcrystalline cellulose by weight; about 10-30%, about 15-25%, about 20-22%, or about 21.5% of calcium phosphate dibasic dihydrate by weight; about 2.5-10%, about 4-7.5%, or about 5% hydroxypropyl methylcellulose by weight; and about 0.1-5%, about 0.3-2.5%, or about 0.5% magnesium stearate by weight.

In some embodiments, the dosage form contains reboxetine free base, such as about 4-6 mg, about 4.5-5.5 mg, or about 5 mg of reboxetine free base, or a molar equivalent amount of a salt form of reboxetine, such as about 5.5-7.5 mg, about 6-7 mg, or about 6.5 mg of reboxetine mesylate; about 50-80%, about 60-70%, about 63-66%, or about 64.6% microcrystalline cellulose by weight; about 10-30%, about 15-25%, about 20-22%, or about 21.5% of calcium phosphate dibasic dihydrate by weight; about 1-10%, about 2-5%, or about 2.5% polyvinylpolypyrrolidone by weight; and about 0.1-5%, about 0.3-2.5%, or about 0.5% magnesium stearate by weight.

In some embodiments, the dosage form contains reboxetine free base, such as about 4-6 mg, about 4.5-5.5 mg, or about 5 mg of reboxetine free base, or a molar equivalent amount of a salt form of reboxetine, such as about 5.5-7.5 mg, about 6-7 mg, or about 6.5 mg of reboxetine mesylate; about 50-80%, about 60-70%, about 63-66%, or about 64.6% microcrystalline cellulose by weight; about 2.5-10%, about 4-7.5%, or about 5% hydroxypropyl methylcellulose by weight; about 1-10%, about 2-5%, or about 2.5% polyvinylpolypyrrolidone by weight; and about 0.1-5%, about 0.3-2.5%, or about 0.5% magnesium stearate by weight.

In some embodiments, the dosage form contains reboxetine free base, such as about 4-6 mg, about 4.5-5.5 mg, or about 5 mg of reboxetine free base, or a molar equivalent amount of a salt form of reboxetine, such as about 5.5-7.5 mg, about 6-7 mg, or about 6.5 mg of reboxetine mesylate; about 10-30%, about 15-25%, about 20-22%, or about 21.5% of calcium phosphate dibasic dihydrate by weight; about 2.5-10%, about 4-7.5%, or about 5% hydroxypropyl methylcellulose by weight; about 1-10%, about 2-5%, or about 2.5% polyvinylpolypyrrolidone by weight; and about 0.1-5%, about 0.3-2.5%, or about 0.5% magnesium stearate by weight.

In some embodiments, the composition comprises about 2% to about 4% reboxetine by weight, or about 2-4%, about 2-2.5%, about 2.5-3%, about 3-3.5%, about 3.5-4%, about 2-3%, about 3-4% reboxetine free base by weight, or a molar equivalent amount of a salt form of reboxetine.

Additional examples of suitable dosage form compositions are listed in Table 1 below.

TABLE 1

| | |
|---|---|
| Ingredient | % w/w |

INTRA-GRANULAR

| | |
|---|---|
| Reboxetine | 3-4 |
| Diluent | 75-95 |
| Binder | 2.5-7.5 |
| Disintegrant | 1-5 |
| Total Intra-Granular Composition | 90-99 |

EXTRA-GRANULAR

| | |
|---|---|
| Glidant | 0.01-1 |
| Disintegrant | 1-5 |
| Lubricant | 0.1-1 |
| Total Extra-Granular Composition | 1-10 |

FILM COAT

| | |
|---|---|
| Blend of polymer, plasticizer, and pigment | 2-5 |

TABLE 2

| | |
|---|---|
| Ingredient | % w/w |

INTRA-GRANULAR

| | |
|---|---|
| Reboxetine | 3-4 |
| Diluent 1 | 60-70 |
| Diluent 2 | 15-25 |
| Binder | 2.5-7.5 |
| Disintegrant | 1-5 |
| Total Intra-Granular Composition | 90-99 |

EXTRA-GRANULAR

| | |
|---|---|
| Glidant | 0.01-1 |
| Disintegrant | 1-5 |
| Lubricant | 0.1-1 |
| Total Extra-Granular Composition | 1-10 |

FILM COAT

| | |
|---|---|
| Blend of polymer, plasticizer, and pigment | 2-5 |

TABLE 3

| | | |
|---|---|---|
| Ingredient | Function | % w/w |

INTRA-GRANULAR

| | | |
|---|---|---|
| Reboxetine Mesylate | Active | 3-4 |
| Microcrystalline Cellulose | Diluent | 60-70 |
| Calcium Phosphate Dibasic Dihydrate | Diluent | 15-25 |
| Hydroxypropyl methylcellulose | Binder | 2.5-7.5 |
| Polyvinylpolypyrrolidone | Disintegrant | 1-5 |
| Total Intra-Granular Composition | | 90-99 |

EXTRA-GRANULAR

| | | |
|---|---|---|
| Silicone Dioxide Colloidal | Glidant | 0.01-1 |
| Polyvinylpolypyrrolidone | Disintegrant | 1-5 |
| Magnesium Stearate | Lubricant | 0.1-1 |
| Total Extra-Granular Composition | | 1-10 |

FILM COAT

| | | |
|---|---|---|
| Blend of polymer, plasticizer, and pigment | Film coating | 2-5 |

Some dosage forms may comprise a uniform blend of reboxetine and a pharmaceutically acceptable excipient. In some embodiments, a pharmaceutical composition comprises a uniform blend of reboxetine and a pharmaceutically acceptable excipient. The excipient may be, for example, a diluent, a binder, a disintegrant, a glidant, a lubricant, or a combination thereof. In some embodiments, a pharmaceutical composition or a dosage form may comprise a uniform blend of reboxetine a diluent, a binder, and a disintegrant.

A uniform blend includes a blend where samples taken from the powder blender have substantially the same amount of reboxetine. In some embodiments:
when 1 sample is tested from each of 10 locations, such as 10 random locations, in the powder blender where blending occurs, the relative standard deviation (relative standard deviation=[(standard deviation)/(mean)]×100%) of the content of reboxetine for the 10 samples, is 5% or less, 4% or less, or 3% or less; or
a) when 3 samples are tested from each of 10 locations, such as 10 random locations, in the powder blender where blending occurs, the relative standard deviation (relative standard deviation=[(standard deviation)/(mean)]×100%) of the content of reboxetine for the 10 samples, is 7% or less, 6% or less, or 5% or less;

In some embodiments, when 10 units of a dosage form, such as 10 tablets, are tested, such as randomly tested, from a single batch, the relative standard deviation (relative standard deviation=[(standard deviation)/(mean)]×100%) of the content of reboxetine for the 10 samples, is 5% or less, 4% or less, or 3% or less.

A composition described herein may be sterilized.

Unless otherwise indicated, any reference to a compound herein, such as reboxetine, by structure, name, or any other means, includes pharmaceutically acceptable salts; alternate solid forms, such as polymorphs, solvates, hydrates, etc.; tautomers; or any other chemical species that may rapidly convert to a compound described herein under conditions in which the compounds are used as described herein.

Reboxetine has at least two polymorph forms: Polymorph 1 and Polymorph 2.

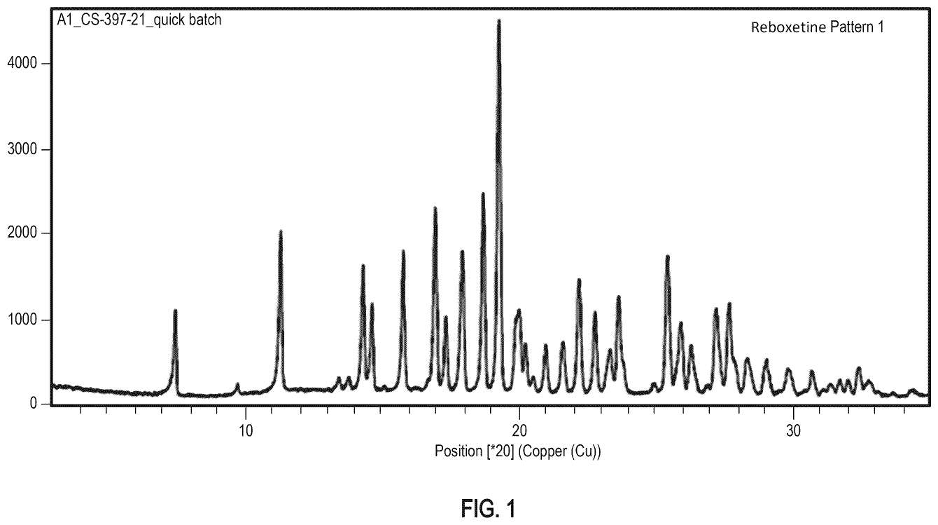

Polymorph 1 has an X-Ray powder diffractogram (XRPD) depicted in FIG. 1. The XRPD peaks that are specific to Polymorph 1 are located at (° 2θ): 7.63, 9.88, 11.45, 14.43, 14.73, 15.89, 17.05, 17.43, 18.04, 18.80, 19.36, and 25.80.

Some embodiments include a polymorph of reboxetine having an XRPD peak located at a position (° 2θ) of 7.63.

Some embodiments include a polymorph of reboxetine having an XRPD peak located at a position (° 2θ) of 9.88.

Some embodiments include a polymorph of reboxetine having an XRPD peak located at a position (° 2θ) of 11.45.

Some embodiments include a polymorph of reboxetine having an XRPD peak located at a position (° 2θ) of 14.43.

Some embodiments include a polymorph of reboxetine having an XRPD peak located at a position (° 2θ) of 14.73.

Some embodiments include a polymorph of reboxetine having an XRPD peak located at a position (° 2θ) of 15.89.

Some embodiments include a polymorph of reboxetine having an XRPD peak located at a position (° 2θ) of 17.05.

Some embodiments include a polymorph of reboxetine having an XRPD peak located at a position (° 2θ) of 17.43.

Some embodiments include a polymorph of reboxetine having an XRPD peak located at a position (° 2θ) of 18.04.

Some embodiments include a polymorph of reboxetine having an XRPD peak located at a position (° 2θ) of 18.80.

Some embodiments include a polymorph of reboxetine having an XRPD peak located at a position (° 2θ) of 19.36.

Some embodiments include a polymorph of reboxetine having an XRPD peak located at a position (° 2θ) of 25.80.

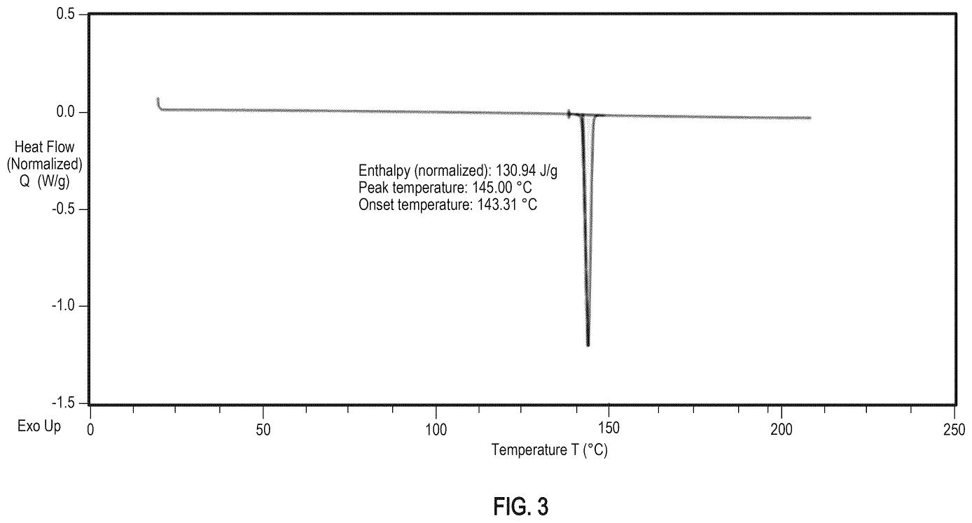

Polymorph 1 has a Differential Scanning calorimetry (DSC) thermogram, at a 1 ° C/min heating rate that is depicted in FIG. 3. The DSC thermogram has an endothermic peak at about 145 ° C, about 145.0 ° C, or about 145.00 ° C

Some embodiments include a polymorph of reboxetine having DSC thermogram which has an endothermic peak at about 145 ° C, about 145.0 ° C, or about 145.00 ° C

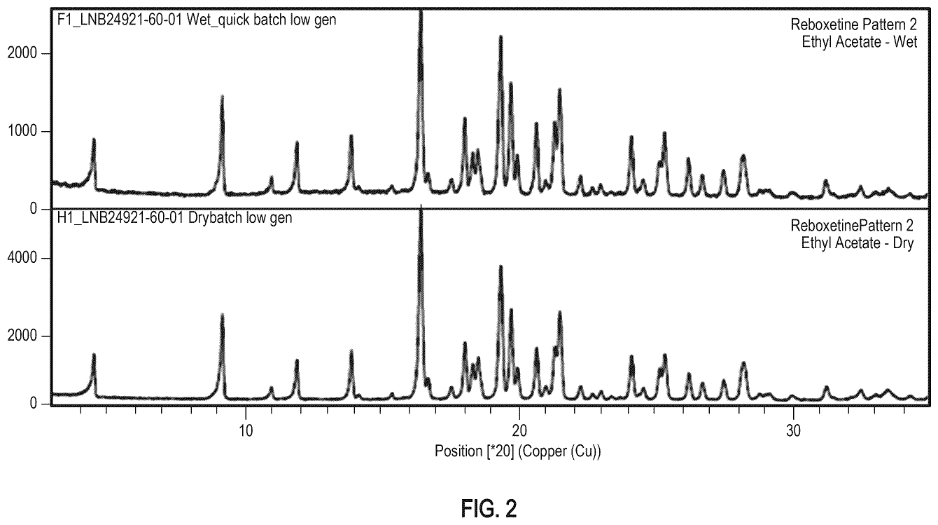

Polymorph 2 has an XRPD depicted in FIG. 2. The peaks that are specific to Polymorph 1 are located at (° 2θ): 4.71, 9.34, 11.12, 12.04, 14.01, 14.28, 15.48, 16.52, 16.79, 17.63, 18.13, 18.40, 19.44, 19.78, 20.65, 24.15, and 26.72.

Some embodiments include a polymorph of reboxetine having an XRPD peak located at a position (° 2θ) of 4.71.

Some embodiments include a polymorph of reboxetine having an XRPD peak located at a position (° 2θ) of 9.34.

Some embodiments include a polymorph of reboxetine having an XRPD peak located at a position (° 2θ) of 11.12.

Some embodiments include a polymorph of reboxetine having an XRPD peak located at a position (° 2θ) of 12.04.

Some embodiments include a polymorph of reboxetine having an XRPD peak located at a position (° 2θ) of 14.01.

Some embodiments include a polymorph of reboxetine having an XRPD peak located at a position (° 2θ) of 14.28.

Some embodiments include a polymorph of reboxetine having an XRPD peak located at a position (° 2θ) of 15.48.

Some embodiments include a polymorph of reboxetine having an XRPD peak located at a position (° 2θ) of 16.52.

Some embodiments include a polymorph of reboxetine having an XRPD peak located at a position (° 2θ) of 16.79.

Some embodiments include a polymorph of reboxetine having an XRPD peak located at a position (° 2θ) of 17.63.

Some embodiments include a polymorph of reboxetine having an XRPD peak located at a position (° 2θ) of 18.13.

Some embodiments include a polymorph of reboxetine having an XRPD peak located at a position (° 2θ) of 18.40.

Some embodiments include a polymorph of reboxetine having an XRPD peak located at a position (° 2θ) of 19.44.

Some embodiments include a polymorph of reboxetine having an XRPD peak located at a position (° 2θ) of 19.78.

Some embodiments include a polymorph of reboxetine having an XRPD peak located at a position (° 2θ) of 20.65.

Some embodiments include a polymorph of reboxetine having an XRPD peak located at a position (° 2θ) of 24.15.

Some embodiments include a polymorph of reboxetine having an XRPD peak located at a position (° 2θ) of 26.72.

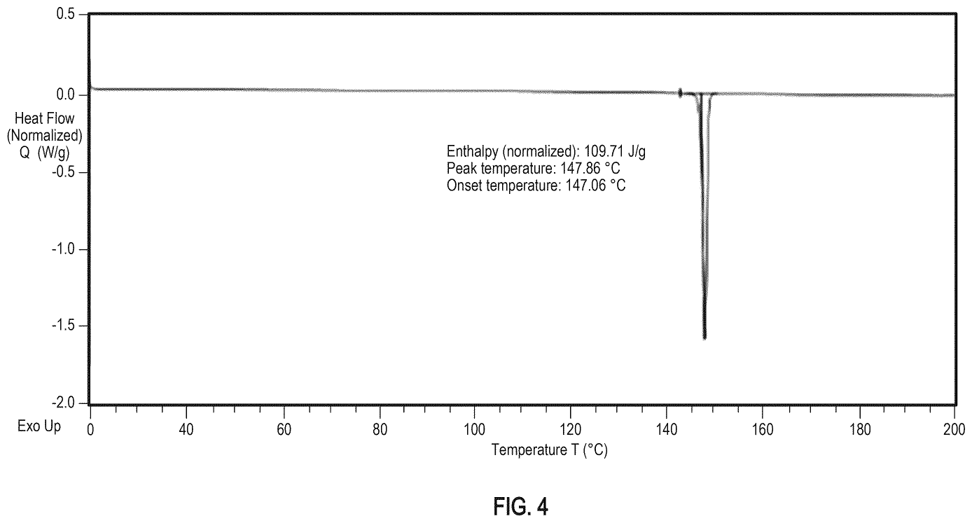

Polymorph 2 has a DSC thermogram, at a 1 ° C/min heating rate that is depicted in FIG. 4. The DSC thermogram has an endothermic peak at about 148 ° C, about 147.9 ° C, or about 147.85 ° C.

Some embodiments include a polymorph of reboxetine having DSC thermogram which has an endothermic peak at about 148 ° C, about 147.9 ° C, or about 147.85 ° C

Some dosage forms containing reboxetine may contain unacceptable levels of nitroso impurities. 2-((2-Ethoxyphenoxy)(phenyl)methyl)-4-nitrosomorpholine below may be used to determine the quality of a pharmaceutical composition comprising reboxetine. For example, a sample of a dosage form may be analyzed using a chromatographic method, such as high-performance liquid chromatography (HPLC) or gas chromatography (GC). A sample of 2-((2-ethoxyphenoxy)(phenyl)methyl)-4-nitrosomorpholine may also be analyzed using a chromatographic method, such as HPLC or GC. This may be useful, for example, for determining the retention time of nitroso impurities, and for determining the sensitivity of the detector or a chromatographic instrument to a nitroso compound. Thus, one method may comprise comparing a chromatographic result obtained from the pharmaceutical composition to a chromatographic result obtained from 2-((2-ethoxyphenoxy)(phenyl)methyl)-4-nitrosomorpholine.

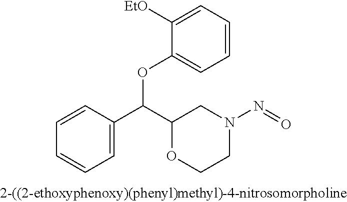 [SMILES: CCOc1ccccc1OC(c1ccccc1)C1CN(N=O)CCO1]

In some embodiments, a pharmaceutical composition or a dosage form may contain less than 15 ppm of 2-((2-ethoxyphenoxy)(phenyl)methyl)-4-nitrosomorpholine, such as less than 13 ppm, about 0.5-13 ppm, about 2-5 ppm, about 0.5-2 ppm, about 2-3 ppm, about 3-4 ppm, about 4-5 ppm, about 5-6 ppm, about 6-8 ppm, about 8-10 ppm, or about 10-13 ppm, of 2-((2-ethoxyphenoxy)(phenyl)methyl)-4-nitrosomorpholine.

The terms “treating” or “treatment” broadly includes any kind of treatment activity, including the diagnosis, cure, mitigation, or prevention of disease in man or other animals, or any activity that otherwise affects the structure or any function of the body of man or other animals. In some embodiments, the mammal being treated is a human being. In some embodiments, the mammal being treated is a non-human mammal, such as a dog, a cat, a mouse, a rat, a rabbit, a monkey, a horse, a pig, etc.

A composition described herein may be used to treat a condition such as a nervous system disorder, including an addictive disorder (including those due to alcohol, nicotine, and other psychoactive substances), a withdrawal syndrome, an adjustment disorder (including depressed mood, anxiety, mixed anxiety and depressed mood, disturbance of conduct, and mixed disturbance of conduct and mood), depression (including major depressive disorder, alone or in combination with other antidepressants), an age-associated learning or mental disorder (including Alzheimer's disease), anorexia nervosa apathy, an attention-deficit (or another cognitive) disorder due to general medical conditions, attention-deficit hyperactivity disorder (ADHD), bipolar disorder, bulimia nervosa, chronic fatigue syndrome, chronic or acute stress, chronic pain, conduct disorder, cyclothymic disorder, depression (including adolescent depression and minor depression), dysthymic disorder, fibromyalgia and other somatoform disorders (including somatization disorder, conversion disorder, pain disorder, hypochondriaism, body dysmorphic disorder, undifferentiated somatoform disorder, and somatoform NOS), generalized anxiety disorder (GAD), incontinence (i.e., stress incontinence, genuine stress incontinence, and mixed incontinence), stress urinary incontinence, an inhalation disorder, an intoxication disorders (alcohol addiction), mania, migraine headaches, obesity (e.g., reducing the weight of obese or overweight patients), an obsessive compulsive disorder or a related spectrum disorder, oppositional defiant disorder, panic disorder, peripheral neuropathy, post-traumatic stress disorder, premenstrual dysphoric disorder (i.e., premenstrual syndrome and late luteal phase dysphoric disorder), a psychotic disorder (including schizophrenia, negative symptoms of schizophrenia, schizoaffective or schizophreniform disorder, either alone or as an adjuvant therapy), seasonal affective disorder, a sleep disorder (such as narcolepsy, including narcolepsy with cataplexy, or enuresis), social phobia (including social anxiety disorder), a specific developmental disorder, selective serotonin reuptake inhibition (SSRI) “poop out” syndrome (i.e., wherein a patient who fails to maintain a satisfactory response to SSRI therapy after an initial period of satisfactory response), TIC disorders (e.g., Tourette's Disease), post-shingles pain, painful diabetic peripheral neuropathy, postherpetic neuralgia, syncope, and/or vasovagal syncope, etc.

Specific dosages maybe adjusted depending on conditions of disease, the age, body weight, general health conditions, sex, and diet of the subject, dose intervals, administration routes, excretion rate, and combinations of drugs.

In some embodiments, a human patient is treated with a dosage form or composition described herein, such as a dosage form of Table 1, Table 2, or Table 3, e.g., containing about 6-7 or about 6.5 mg of reboxetine mesylate, or a molar equivalent of another salt form, or a molar equivalent amount of the free base form (e.g., 5 mg of reboxetine free base), e.g., once daily, to treat narcolepsy, such as Type I narcolepsy, or narcolepsy with cataplexy.

A person may have Type 1 narcolepsy if Criteria A and B are met:
A. The patient has daily periods of irrepressible need to sleep or daytime lapses into sleep occurring for at least 3 months
B. The presence of one or both of the following:
   1. Cataplexy (as defined under Essential Features) and a mean sleep latency of <8 minutes and ≥2 Sleep-Onset REM Periods (SOREMPs) on a Mean Sleep Latency Test (MSLT) performed according to standard techniques. A SOREMP (within 15 minutes of sleep onset) on the preceding laboratory-based polysomnography (PSG) may replace one of the SOREMPs on the MSLT
   2. CSF hypocretin-1 concentrations measured by immunoreactivity either <110 μg/mL or <⅓ of mean values obtained in normal subjects with the same assay.

### Non-Limiting Examples

### Specifically Contemplated Embodiments

The following are examples of embodiments that are specifically contemplated by the inventor:

Embodiment 1. A polymorph of reboxetine having an X-Ray power diffractogram (XRPD) with a peak located at a position (° 2θ) of 7.63, 9.88, 11.45, 14.43, 14.73, 15.89, 17.05, 17.43, 18.04, 18.80, 19.36, 25.80, or a combination thereof.

Embodiment 2. A dosage form comprising the polymorph of embodiment 1 and less than 13 ppm of a compound represented by a formula:

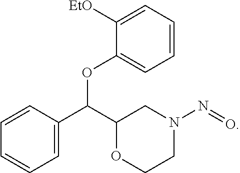 [SMILES: CCOc1ccccc1OC(c1ccccc1)C1CN(N=O)CCO1]

Embodiment 3. The dosage form of any preceding embodiment, further comprising microcrystalline cellulose.

Embodiment 4. The dosage form of any preceding embodiment, further comprising calcium phosphate.

Embodiment 5. The dosage form of any preceding embodiment, further comprising hydroxypropyl methylcellulose.

Embodiment 6. The dosage form of any preceding embodiment, further comprising a polyvinylpyrrolidone.

Embodiment 7. The dosage form of any preceding embodiment, further comprising a crosslinked polyvinylpyrrolidone.

Embodiment 8. The dosage form of any preceding embodiment, comprising about 3% to about 4% reboxetine by weight.

Embodiment 9. The dosage form of any preceding embodiment, comprising about 60% to about 70% of a microcrystalline cellulose by weight.

Embodiment 10. The dosage form of any preceding embodiment, comprising about 15% to about 25% of a calcium phosphate by weight.

Embodiment 11. The dosage form of any preceding embodiment, comprising about 2.5% to about 7.5% of a hydroxypropyl methylcellulose by weight.

Embodiment 12. The dosage form of any preceding embodiment, comprising about 1% to about 5% of a polyvinylpolypyrrolidone by weight.

Embodiment 13. A compound represented by a formula:

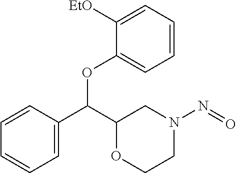 [SMILES: CCOc1ccccc1OC(c1ccccc1)C1CN(N=O)CCO1]

Embodiment 14. A method of determining the quality of a pharmaceutical composition comprising reboxetine, comprising comparing a chromatographic result obtained from the pharmaceutical composition to a chromatographic result obtained from the compound of embodiment 13.

Embodiment 15. A dosage form comprising reboxetine and less than 13 ppm of a compound represented by a formula:

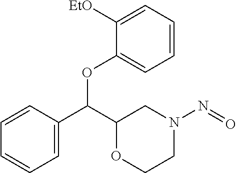 [SMILES: CCOc1ccccc1OC(c1ccccc1)C1CN(N=O)CCO1]

Embodiment 16. The dosage form of embodiment 15, comprising about 60% to about 70% of a microcrystalline cellulose by weight.

Embodiment 17. The dosage form of any preceding embodiment, comprising about 15% to about 25% of a calcium phosphate by weight.

Embodiment 18. The dosage form of any preceding embodiment, comprising about 2.5% to about 7.5% of a hydroxypropyl methylcellulose by weight.

Embodiment 19. The dosage form of any preceding embodiment, comprising about 1% to about 5% of a polyvinylpolypyrrolidone by weight.

Embodiment 20. A pharmaceutical composition comprising a uniform blend of reboxetine and a pharmaceutically acceptable excipient.

Embodiment 21. The pharmaceutical composition of embodiment 20, wherein the pharmaceutically acceptable excipient comprises microcrystalline cellulose.

Embodiment 22. The pharmaceutical composition of any preceding embodiment, wherein the pharmaceutically acceptable excipient comprises calcium phosphate.

Embodiment 23. The pharmaceutical composition of any preceding embodiment, wherein the pharmaceutically acceptable excipient comprises hydroxypropyl methylcellulose.

Embodiment 24. The pharmaceutical composition of any preceding embodiment, wherein the pharmaceutically acceptable excipient comprises a polyvinylpolypyrrolidone.

Embodiment 25. The pharmaceutical composition of any preceding embodiment, wherein the composition comprises about 3% to about 4% reboxetine by weight.

Embodiment 26. The pharmaceutical composition of any preceding embodiment, wherein the pharmaceutically acceptable excipient comprises about 60% to about 70% of a microcrystalline cellulose by weight.

Embodiment 27. The pharmaceutical composition of any preceding embodiment, wherein the pharmaceutically acceptable excipient comprises about 15% to about 25% of a calcium phosphate by weight.

Embodiment 28. The pharmaceutical composition of any preceding embodiment, wherein the pharmaceutically acceptable excipient comprises about 2.5% to about 7.5% of a hydroxypropyl methylcellulose by weight.

Embodiment 29. The pharmaceutical composition of any preceding embodiment, wherein the pharmaceutically acceptable excipient comprises about 1% to about 5% of a polyvinylpolypyrrolidone by weight.

As used herein in the specification and in the claims, the articles “a” and “an” are used to refer to one or to more than one (i.e., to at least one) of the grammatical object of the article unless the context clearly indicates otherwise. By way of example, “an element” means one element or more than one element.

As used herein in the specification and in the claims, the phrase “and/or,” should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of,” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

Wherever the phrase “for example,” “such as,” “including,” and the like are used herein, the phrase “and without limitation” is understood to follow unless explicitly stated otherwise. Similarly, “an example,” “exemplary,” and the like are understood to be non-limiting.

1. A dosage form comprising 4.5 mg of reboxetine mesylate and a pharmaceutically acceptable excipient.

2. The dosage form of claim 1, wherein the pharmaceutically acceptable excipient comprises microcrystalline cellulose.

3. The dosage form of claim 1, wherein the pharmaceutically acceptable excipient comprises calcium phosphate.

4. The dosage form of claim 1, wherein the pharmaceutically acceptable excipient comprises hydroxypropyl methylcellulose.

5. The dosage form of claim 1, wherein the pharmaceutically acceptable excipient comprises a polyvinylpyrrolidone.
